(19) United States
(12) Patent Application Publication
(10) Pub. No.: US 2021/0001024 A1
(43) Pub. Date: Jan. 7, 2021

(54) BREASTSHIELD UNIT

(71) Applicant: MEDELA HOLDING AG, Baar (CH)

(72) Inventors: Martin Thüring, Sins (CH); Sebastian Höner, Thalwil (CH)

(21) Appl. No.: 16/976,071

(22) PCT Filed: Mar. 4, 2019
(86) PCT No.: PCT/EP2019/055240

§ 371 Date: Aug. 26, 2020

(30) Foreign Application Priority Data
Mar. 6, 2018 (EP) 18160184.0

(51) Int. Cl. A61M 1/06; A61M 1/00

(52) U.S. Cl. CPC A61M 1/062; A61M 1/0001; A61M 2205/3379; A61M 2205/3306; A61M 2206/20; A61M 2205/50

(57) ABSTRACT

A breastshield unit of a breast pump for expressing human breastmilk comprises a breastshield (1) for placing onto a human breast, a milk collection container (4) for receiving expressed breastmilk, a breastshield adapter (2) for connecting the breastshield (1) to the milk collection container (4), and at least one sensor (6, 7) for detecting the breastmilk. From the breastshield, a milk channel extends through the breastshield adapter into the milk collection container. A siphon is arranged in the milk channel, and the at least one sensor (6, 7) for detecting the breastmilk is arranged downstream from the siphon in the direction of flow of the breastmilk. The breastshield unit permits optimal measurement of properties of the expressed breastmilk.

### TECHNICAL FIELD

The present invention relates to a breastshield unit of a breast pump for expressing human breastmilk, to a sensor unit of a breast pump, and to a method for determining at least one property of human breastmilk during the expression process.

### PRIOR ART

In order to express human breastmilk, a breastshield is placed onto the mother's breast, said breastshield being connected to a vacuum pump in order to apply a varying negative pressure to the breast. The negative pressure preferably varies cyclically or according to a predefined or empirically evaluated suction curve.

From the breastshield, the expressed milk passes through a milk channel into a milk collection container. In order to keep the dead volume as small as possible during the expression process, a milk channel running from the breastshield to the milk collection container is usually provided with a check valve which opens in order to admit the expressed milk into the milk collection container. This check valve is preferably arranged at a breastshield adapter which connects the breastshield to the milk collection container and also, usually, the breastshield to the vacuum pump or to a vacuum tube leading to the vacuum pump. The check valve can be arranged on a valve head which is connected in a secure or detachable manner to the breastshield adapter, the latter also being referred to as coupling part. Breastshield and adapter are usually two separate components. However, they can also be formed together in one piece.

A device of this kind is known from WO 2014/161099 A1, for example. The breastshield unit described there has a throughflow sensor which detects the change of a valve flap of the check valve, such that conclusions can be drawn regarding the throughflow of the milk from the breastshield into the milk collection container.

US 2016/0220743 A1 discloses an adapter with sensors for detecting the individual drops of the expressed milk. The sensors are arranged upstream from the check valve in the direction of flow of the milk.

US 2015/0283311 A1 describes various sensors which are arranged in the neck or in the bottom of the breastmilk bottle.

US 2017/0021068 A1 discloses a fluid sensor which is connected to the milk collection container.

Measurements by sensors arranged in the milk flow are often too imprecise since, depending on the mother and on the expression phase, the milk flows only in drops or flows more abundantly. Moreover, the milk is mixed with different quantities of air, which likewise adversely affects the accuracy of the measurement of a throughflow quantity of milk.

Sensors that measure the milk quantity already received in the milk collection container are usually unsuitable for the measurement of small quantities of milk, e.g. quantities of up to 30 ml.

A further factor preventing precise measurements of the flow of milk, or of the expressed quantity of milk, is the media separation membrane. A media separation membrane is used to protect the breast pump from contamination. It separates the region in which the vacuum is generated from the region in which the milk flows, wherein it transmits the vacuum generated by the breast pump into the interior of the breastshield. In particular, sensors located on flaps or check valves are influenced by these media separation membranes, since the behaviour of the check valve is influenced by the movement of the media separation membrane.

Devices for measuring quantities of urine are also known which use a siphon arrangement. Such devices are described in DE 41 06 995 A1, DE 41 14 933 A1, U.S. Pat. Nos. 3,641,818, 3,919,455, 5,656,027, GB 2 031 158 and WO 2010/149708 A1.

### DISCLOSURE OF THE INVENTION

It is an object of the invention to provide a breastshield unit of a breast pump for expressing human breastmilk, which permits optimization of the measurement of the expressed milk.

This object is achieved by a breastshield unit having the features of Patent claim 1, by a sensor unit of a breast pump according to claim 15, and by a method according to claim 16 for determining at least one property of human breastmilk.

The breastshield unit, according to the invention, of a breast pump for expressing human breastmilk comprises a breastshield for placing onto a human breast, a milk collection container for receiving expressed breastmilk, a breastshield adapter for connecting the breastshield to the milk collection container, and at least one sensor for detecting the breastmilk. A milk channel extends from the breastshield through the breastshield adapter into the milk collection container. The milk channel defines a direction of flow of the breastmilk. According to the invention, a siphon is arranged in the milk channel, and the at least one sensor for detecting the breastmilk is arranged downstream from the siphon in the direction of flow of the breastmilk.

A siphon within the meaning of this application also comprises loops and other geometries that lead to a section of the milk channel being emptied in batches, i.e. in a pulsed manner.

By virtue of its damming region, the siphon arrangement has a settling effect on the irregular inflow of milk. The siphon arrangement additionally has the advantage that only the milk collects in the damming region, and therefore the air/milk mixture that has a disruptive effect on the measurement in other systems is separated out.

It is moreover advantageous that individual columns of milk pass the at least one downstream sensor in batches. In this way, a clear separation layer between air and milk is discernible and detectable. The column of milk is also called the milk volume or milk batch.

The detection of the air/milk separation layer at the start of the milk column passing the sensor and the detection of the milk/air separation layer at the end of the milk column passing the sensor, and the evaluation of the signals thereby obtained as a function of time, provide information on the milk throughflow quantity and milk throughflow rate during a defined time interval. The total number of the milk columns provides information on the milk quantity. The number of milk columns during a defined time interval provides information on the expressed quantity of milk during this defined time. It is thus possible to determine the quantity of milk and the throughflow rate during the entire duration of pumping, likewise the quantity of milk and the throughflow rate during individual pump phases.

The flow behaviour of the milk can likewise be detected by virtue of the clear separation of milk and air. This flow behaviour provides information, for example, on the viscosity and thus the composition of the milk. It is thus possible to draw conclusions concerning the macronutrient content of the milk, for example the fat content, proteins and lactose and/or combined forms thereof.

A valve is preferably present which closes and opens the milk channel during the expression of the breastmilk. The siphon is preferably arranged downstream from the valve in the direction of flow of the breastmilk.

This breastshield unit permits a measurement of the quantity of milk in a manner that is not adversely affected by the changing negative pressure applied or by the vacuum applied. The valve, which is preferably a one-way valve or a check valve, separates the expression region from the measurement region.

Moreover, any media separation membrane that is present does not therefore influence the measurement, since it influences only the expression region.

Depending on the property of the milk that is to be measured, one sensor is present, or two or more sensors are needed. If two or more sensors are present, they are preferably arranged in succession in the direction of flow.

In a preferred embodiment, the breastshield unit comprises a signal evaluation module for evaluating signals of the at least one sensor. The signal evaluation module supplies a profile of the signals as a function of time, as a result of which at least one of the following variables can be determined: throughflow quantity, throughflow rate, throughflow velocity, macronutrients, fat content. The signal evaluation module can be arranged in the region of the breastshield, of the breastshield adapter, of the milk collection container or of another component located in this region. However, it can also be arranged spaced apart from these components, for example inside a housing that also accommodates the breast pump assembly and/or the control of the breast pump.

In a preferred embodiment, the milk channel comprises a widened region between valve and siphon. This widened region serves for settling the milk flowing in through the valve, i.e. for the first separation of the air/milk mixture and for the avoidance of eddies. The widened region is preferably a funnel narrowing in the direction of flow.

The milk channel preferably has a constant diameter along the length of the siphon.

Instead of a single lumen of the milk channel, it is also possible to use two or more lumens, i.e. the milk can also flos in a divided form through different lumens. This is advantageous, for example, when different sensors are arranged in the individual lumens and detect different properties of the breastmilk. In this way, the measurements do not influence each other.

The sensor and the siphon can be arranged in the region of the breastshield, of the breastshield adapter or of the milk collection container. In preferred embodiments, however, a sensor unit is present which is preferably arranged between the breastshield adapter and the milk collection container, wherein the sensor unit comprises the siphon of the milk channel and the at least one sensor. The sensor unit is preferably a separate component which is connectable to the breastshield adapter and to the milk collection container preferably in such a way as to be releasable therefrom without destruction. In other embodiments, however, it is preferably connected in part or as a whole component fixedly to the breastshield adapter and/or to the milk collection container.

The milk channel can be formed by a hose or a tube at least in the region of the siphon.

In preferred embodiments, however, the sensor unit has a main body with a surface and with a cover tightly closing this surface. At least the siphon of the milk channel is formed by a groove which is configured in the surface and/or in the cover and which, by the cooperation of the surface with the cover, is tightly closed except for an inlet and an outlet. Such arrangements can be optimally cleaned, since in particular the siphon region is easily accessible over its entire length.

In preferred embodiments, the surface is a radially outwardly oriented surface of the main body. The cover is in this case preferably a lid, which can be placed onto the jacket of the main body, or a sleeve, which can be engaged over the main body.

In other preferred embodiments, the sensor unit has an interior which is open at the top and in which the surface is formed in an undulating shape. The cover is in this case preferably configured as an undulating counterpart.

The sensor unit can be made extremely compact and robust if the main body has a cavity for receiving an electronics unit. The electronics unit preferably has the signal evaluation module. The electronics unit preferably has waterproof protection, such that the main body can be rinsed with water or washed in a wash basin in order to clean the milk channel and in particular the siphon.

The sensor unit preferably has a substantially rotationally symmetrical configuration. It preferably has the shape of a circular cylinder or the shape of a truncated cone. In this way, it can be formed flush with the other components of the breastshield unit and it can be connected to these via thread connections or bayonet connections.

In preferred embodiments, the at least one sensor is at least one optical sensor. However, it is also possible to use other types of sensors, e.g. capacitive sensors, calorimetric sensors, electrochemical or inductive sensors, ultrasound sensors, temperature sensors, sensors for measuring resistance, impedance meters, running time meters, radar and optical sensors such as CCD cameras. In preferred embodiments, more than one sensor is present. Preferably, different types of sensors are present. If a position sensor is present which determines the rotation position of the breastshield unit in space, measured values can be corrected according to the measured position.

The sensor unit according to the invention is part of a breast pump for expressing human breastmilk, wherein the breast pump comprises a breastshield for placing onto a human breast, a milk collection container for receiving expressed breastmilk, a milk channel extending from the breastshield to the milk collection container, and at least one sensor for detecting the breastmilk, wherein the milk channel defines a direction of flow of the breastmilk. The sensor unit comprises a part of the milk channel, wherein this part of the milk channel forms a siphon. Downstream from the siphon in the direction of flow of the breastmilk, the sensor unit comprises the at least one sensor for detecting the breastmilk. A valve is preferably present which closes and opens the milk channel during the expression process.

This sensor unit is preferably arranged in the region of the breastshield and of the milk collection container. However, if breastshield and milk collection container lie far apart from each other, it is preferably arranged either in the region of the breastshield or in the region of the milk collection container. However, it can also be arranged in a region therebetween, i.e. spaced apart from the breastshield and milk collection container, for example in the region of the breast pump housing of the pump assembly.

The method according to the invention for determining at least one property of human breastmilk takes place during expression of the human breastmilk, wherein the breastmilk is expressed by application of a changing negative pressure. The method comprises the following steps, which are carried out in succession, but not necessarily directly after each other:
collecting the breastmilk in a damming region of a siphon region,
emptying the damming region batch by batch via a siphon loop of the siphon region to form a breastmilk column,
forwarding the breastmilk column to at least one sensor,
detecting the breastmilk column, which has been forwarded batch by batch, by means of the at least one sensor, and
evaluating signals of the sensor as a function of time.

The breastmilk is preferably conveyed first through a valve, in particular a check valve, and only then is it collected in the damming region of the siphon region.

Further embodiments are set forth in the dependent claims.

### BRIEF DESCRIPTION OF THE DRAWINGS

Preferred embodiments of the invention are described below with reference to the drawings, which serve only for explanatory purposes and are not to be interpreted as limiting the invention. In the drawings:

FIG. 1 shows a perspective view of a first embodiment of the breastshield unit according to the invention;

FIG. 2 shows a longitudinal section through the breastshield unit according to FIG. 1;

FIG. 3 shows a partial section through a sensor unit of the breastshield unit according to FIG. 1;

FIG. 4 shows a further partial section through the sensor unit of the breastshield unit according to FIG. 1;

FIG. 5 shows a perspective view of an electronics unit of the sensor unit according to FIG. 1;

FIGS. 6a to 6j show schematic views of a siphon of a breastshield unit according to the invention;

FIG. 7a shows a graph in which the signals of two sensors arranged in succession in the direction of flow are plotted as a function of time;

FIG. 7b shows a graph in which the signals of one sensor are plotted as a function of time;

FIG. 8 shows a perspective exploded view of a main body and of a cover of the sensor unit in a second embodiment;

FIG. 9 shows a perspective exploded view of a main body and of a cover of the sensor unit in a third embodiment;

FIG. 10 shows a perspective exploded view of a main body and of a cover of the sensor unit in a fourth embodiment;

FIG. 11 shows a perspective exploded view of a main body and of a cover of the sensor unit in a fifth embodiment;

FIG. 12 shows a perspective exploded view of a main body and of a cover of the sensor unit according to FIGS. 1 to 5;

FIG. 13 shows a perspective exploded view of a main body and of a cover of the sensor unit in a seventh embodiment, and

FIG. 14 shows a longitudinal section through the main body with fitted cover according to FIG. 13.

### DESCRIPTION OF PREFERRED EMBODIMENTS

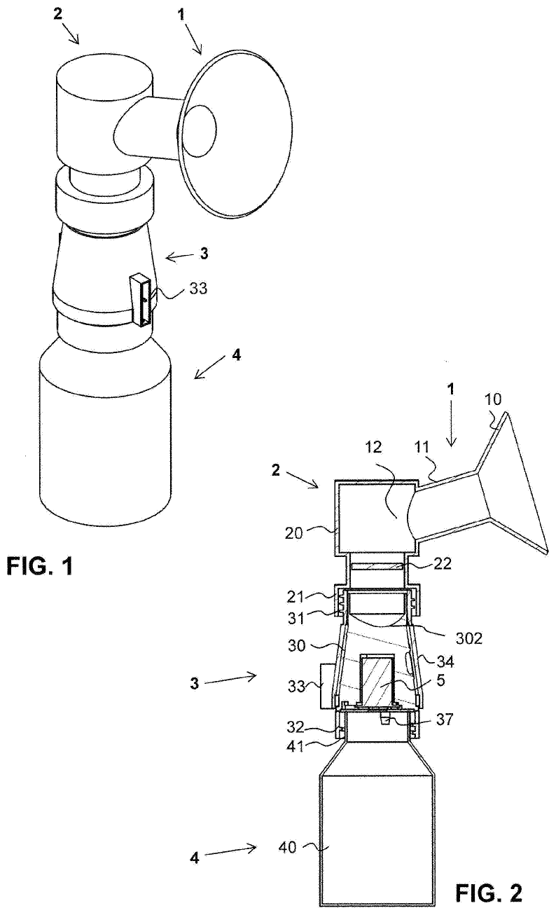

FIGS. 1 to 5 show a first preferred embodiment of the breastshield unit according to the invention. As can be easily seen in FIGS. 1 and 2, it has a breastshield 1, a breastshield adapter 2, a sensor unit 3 and a milk collection container 4.

The breastshield 1 preferably has a funnel-shaped shield 10, which is to be placed sealingly onto a human breast, and an adjoining tubular connector 11. Shield 10 and connector 11 are preferably formed together in one piece. However, they can also be configured as separate components, each composed of one piece or of several pieces. Such breastshields 1 are known in a wide variety of embodiments in the prior art.

The breastshield adapter 2 in this example is formed in one piece with the breastshield 1. However, it is preferably a separate component that can be connected to the breastshield 1 in a releasable manner.

It preferably has a main body 20 with a port (not visible here) for connection to a vacuum source. Depending on the embodiment, the port is a connection element for connection to a suction hose, of which the second end is connected to a housing of a pump assembly of the breast pump. However, the pump assembly of the breast pump can also be arranged directly on the breastshield adapter 2. The pump assembly can be operated manually. However, it is preferably a pump assembly driven by an electric motor. All of these variants are well known from the prior art.

In the breastshield adapter 2, depending on the embodiment, a media separation membrane is arranged between breastshield 1 and the port leading to the vacuum source. Such media separation membranes are likewise very well known, and they are therefore not discussed in any more detail here.

A valve 22 is moreover arranged in the breastshield adapter 2. This valve 22 is preferably a one-way valve, in particular a check valve. In preferred embodiments, it is a flap articulated at one side, a duckbill valve or a centrally suspended valve. The valve 22 divides the inner region of the breastshield unit into an expression region 12 and a measurement region. In the expression region 12, the changing vacuum is applied to the human breast. The measurement region is not directly exposed to these pressure changes. The measurement region is described in more detail below.

The breastshield adapter 2 moreover has an attachment 21 for connecting to a further component, here the sensor unit 3. The connection is preferably releasable without destruction, for the purpose of cleaning the individual components, and is therefore able to be re-established. The connection is preferably effected via a thread or a bayonet catch. For this purpose, the sensor unit 3 has an upper attachment part 31.

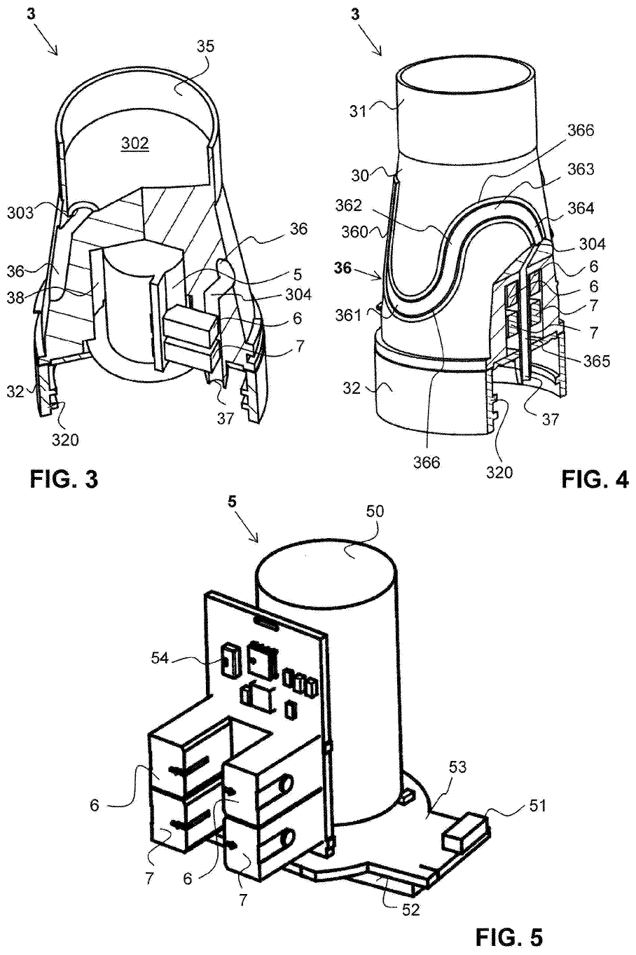

The sensor unit 3 is also preferably connected to the milk collection container 4 in such a way as to be releasable from the latter without destruction. Here too, the connection is preferably effected via a thread or via a bayonet catch. The thread on a lower attachment part 32 of the sensor unit 3 is provided with reference sign 320 in FIGS. 3 and 4. This thread engages around a neck 41 of the milk collection container 4, as can be seen from FIG. 2. The main body of the milk collection container 4 is provided with reference sign 40.

The described components preferably have a substantially rotationally symmetrical design. The sensor unit 3 preferably has a main body 30 having the shape of a truncated cone or of a circular cylinder.

This main body 30 is covered by a cover 34 at least over part of its jacket, in this example over its entire circumference. This can be seen clearly in FIG. 2. The cover 34 seals off the main body 30 in a liquid-tight and airtight manner. The cover 34 is preferably releasable from the main body 30 without destruction, for the purpose of cleaning the components, and can be refitted again.

The main body 30 of the sensor unit 3 preferably has a central cavity 38, which is preferably closed off tightly from the outside, at least in a liquid-tight manner. This can be clearly seen in FIGS. 2 to 4. An electronics unit 5 is arranged in this cavity 38. This electronics unit 5 can be seen clearly in FIG. 5. It has a printed circuit board 53 with a battery 50 or another energy store. A signal evaluation module, here in the form of a microcontroller 52, is arranged on the printed circuit board 53.

The device is preferably switched to an energy-saving mode as soon as the sensor unit 3 is released from the breastshield adapter 2 and/or from the milk collection container 4. For this purpose, a reed switch 51 is preferably present which separates or switches off the current supply as soon as the cover 34 is removed from the main body 30. A holder for a reed switch magnet is provided with reference sign 33 in FIGS. 1 and 2.

Moreover, the electronics unit 5 preferably has a TOF sensor (time-of-flight sensor), which is not visible here. It is preferably located on the underside of the electronics unit and faces towards the interior of the milk collection container 4. By means of this TOF sensor, the filling level of the milk collection container 4 can be measured continuously. This measurement is preferably combined with a measurement of the throughflow, e.g. of the throughflow rate, the throughflow volume or the throughflow quantity.

Sensors are moreover connected to the printed circuit board 53. In this example, a first light barrier 6 and a second light barrier 7 are present, and also an acceleration sensor 54. By means of these or other sensors, said properties of the breastmilk are preferably detected, in particular measured.

A milk channel extends through the entire breastshield unit. The channel extends from the breastshield 1, into which milk is expressed from the human breast, as far as the interior of the main body 40 of the milk collection container 4. The channel is thus formed by the cavities connected to one another in the breastshield unit and leads through the valve 22. In the region of the sensor unit 3, the milk channel, as can be seen clearly in FIGS. 3 and 4, leads through a broad inlet 35 into a funnel structure with an inclined surface 302, which has a small peripheral inlet opening 303. This inlet opening 303 leads to a milk channel section 36 which is located substantially on the outside of the main body 30 and is thus configured as an open groove. It has two loops and forms a siphon. It is provided with a sealing lip 366 on both sides, preferably along its entire length. The abovementioned cover 34 at least covers this milk channel section 36, such that the latter is closed in an airtight and liquid-tight manner except for the inlet opening and an outlet opening 304.

As can be seen in FIGS. 3 and 4, the milk channel section 36 extends from the inlet opening 303 in the interior of the main body 30 to the jacket surface and into a first channel region 360 which extends downwards when the breast pump unit is in its intended state of use. The first channel region 360 merges into a first siphon bend 361, which opens into an upwardly directed second channel region 362. The second channel region 362 is adjoined by a second siphon bend 363, which is in turn adjoined by a downwardly directed third channel region 364. The third channel region 364 merges via an outlet opening 304 into a fourth channel region 365 which extends in the interior of the main body 30 and which ends in an outlet 37 opening downwards into the cavity of the milk collection container.

As can be seen in FIGS. 3 and 4, the first and second light barriers 6, 7 are arranged along the fourth channel region 365. They preferably protrude into the fourth channel region 365 or end flush with the wall of the fourth channel region 365. Other arrangements are possible. The first light barrier 6 is located upstream from the second light barrier 7 in the direction of flow of the milk, which is defined by inlet 35 and outlet 37. The region around the light barriers 6, 7 is of course sealed in an airtight and liquid-tight manner. The milk collection section 36 preferably has a constant cross section, at least in the regions of the first to third channel regions 360, 362, 364 and of the two siphon bends 361, 363.

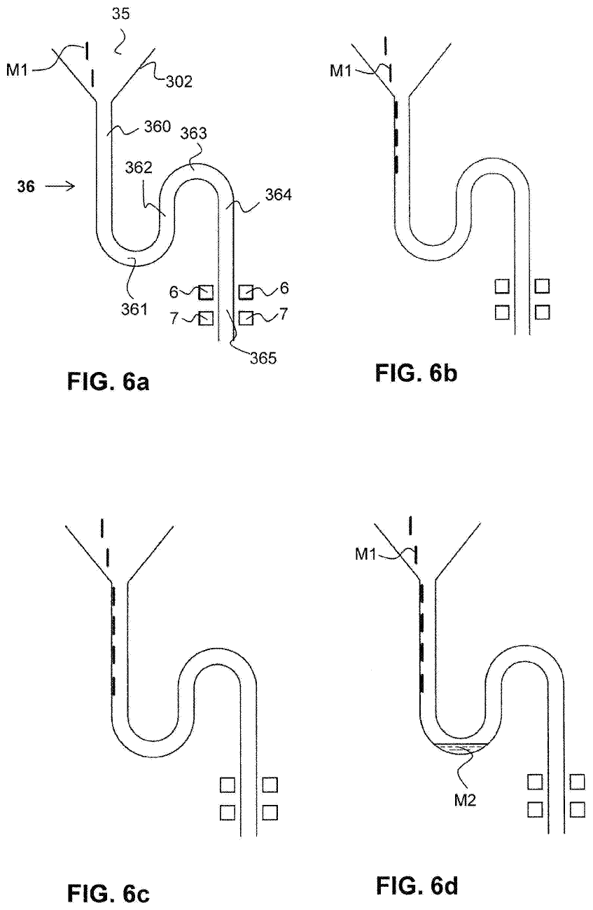

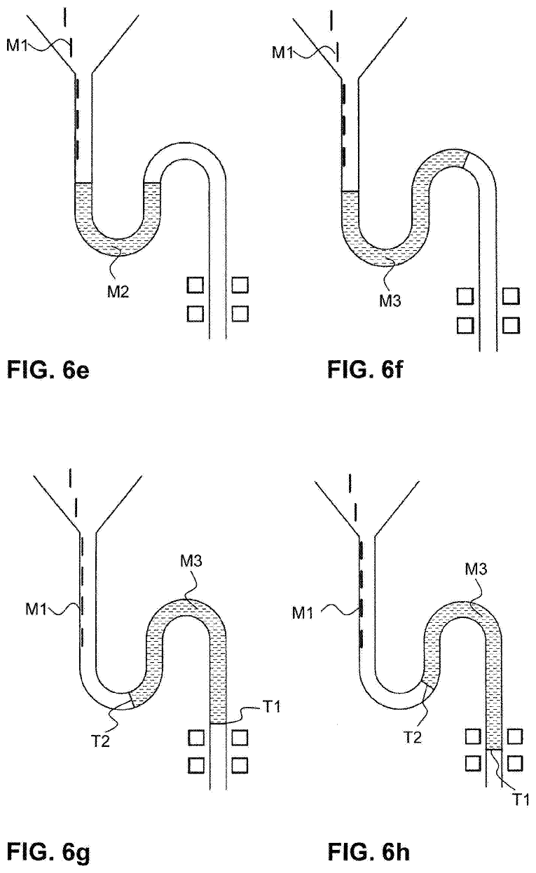

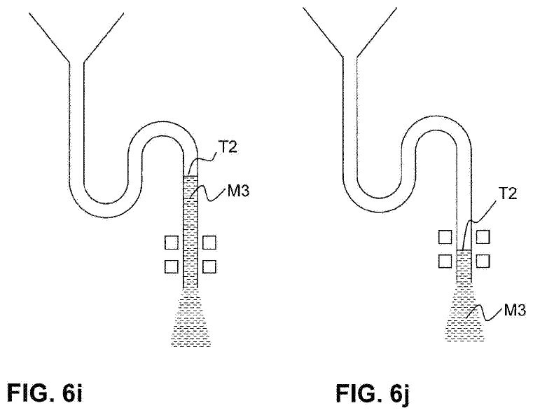

This milk channel section 36, the inlet 35 with the funnel on the inlet side having the inclined surface 302, and the two light barriers 6, 7 are shown schematically once again in FIGS. 6a to 6j. The basic principle of the measurements by means of the siphon is explained with reference to these drawings.

In FIG. 6a, expressed milk M1 passes into the inlet 35. In FIGS. 6b and 6c, further expressed milk M1 follows drop by drop and thus reaches the milk channel section 36, more precisely the first channel region 360. The first siphon bend 361 forms a damming region, where the milk collects to form a milk lake M2. Air from the milk/air mixture already passes into the next region of the milk channel section 36 and is carried off via the outlet 37. This can be see in FIG. 6d. The milk lake M2 then grows ever larger, as is shown in FIG. 1e. It rises along the second channel region 362, until it reaches the upper siphon bend 363 and passes beyond the zenith of the latter. This then triggers the siphon effect, i.e. the dammed-up milk moves as a milk column M3, also called milk volume or milk block, in the direction of the outlet 37. This can be seen clearly in FIGS. 6g to 6j. The milk M1 that is expressed in the meantime again reaches only as far as the first siphon bend 361, where a milk lake M2 again grows in size to form a milk column M3.

The milk column M3 forms two clear separation lines T1, T2 between air and milk. These separation lines T1, T2 can be detected very precisely by means of sensors, for example by means of optical sensors and in particular by means of the light barriers 6, 7 shown here. The signals thus obtained can be processed by means of the signal evaluation module of the electronics system, and various measurements can be performed.

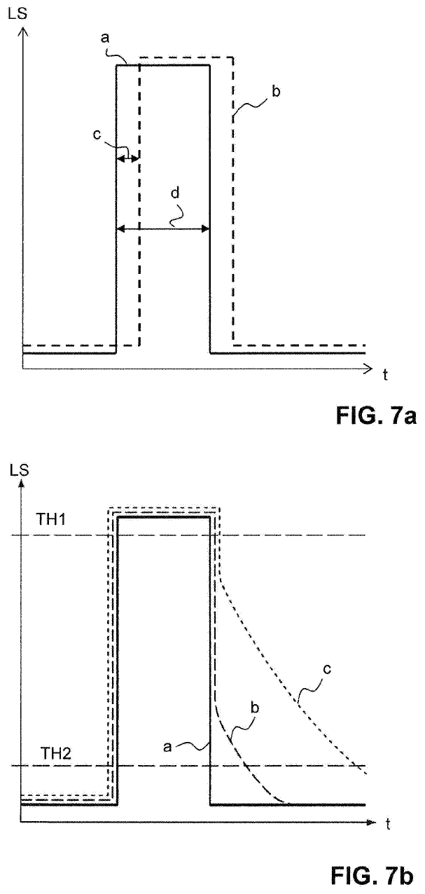

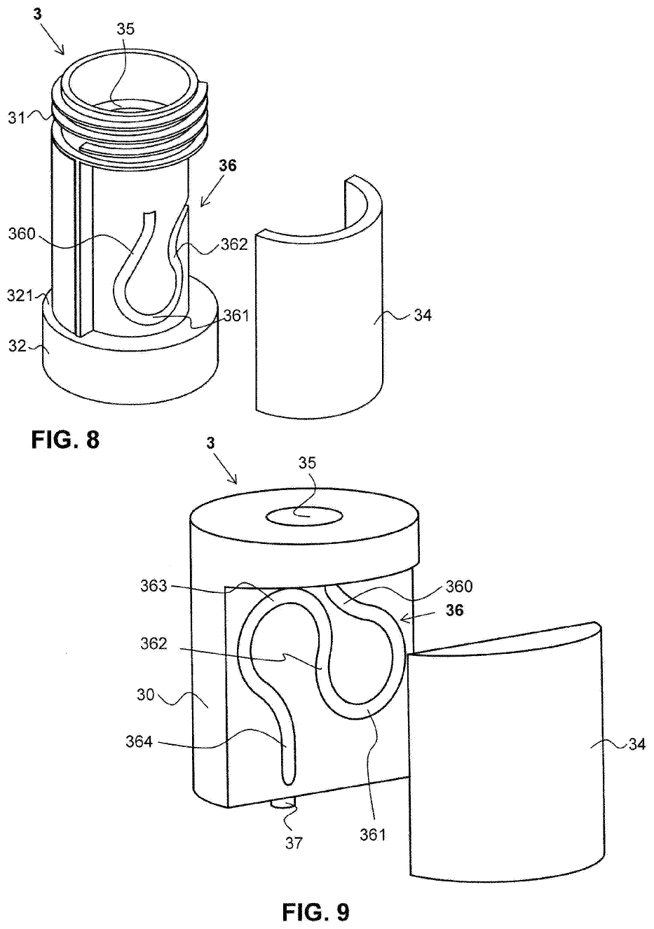

FIG. 7a illustrates, for example, how the two light barriers 6, 7 arranged in succession in the direction of flow can be used to determine the velocity of the throughflow of milk and then the milk volume. Reference sign a designates the signal LS of the first light barrier 6, and reference sign b the signal LS of the second light barrier 7, in relation to the time t. d thus designates the length of time needed for the milk column M3 to run through one of the two light barriers 6, 7. c designates the time lag of the signal, since of course the milk column M3 first passes the first light barrier 6 and only passes the second light barrier 7 after a time delay.

The velocity vᵢ of a column is then:

vᵢ=(distance between the two light barriers): cᵢ

The volume V is obtained from:

Vᵢ=vᵢ*(cross-sectional area of milk channel section)*dᵢ

The total volume V, i.e. the total quantity of expressed breastmilk, is obtained from:

Vₜₒₜₐₗ=Σ Vᵢ

FIG. 7b shows, as a further example, the determination of the macronutrient value. The latter also serves as an indicator of the fat content of the milk, since the viscosity, i.e. the flow behaviour of the milk, is substantially influenced by the fat content.

FIG. 7b shows the signal profile of an individual light barrier, e.g. of the first light barrier 6, wherein a first and a second threshold value TH1, TH2 of the light barrier signal are used for further evaluation. The curve a indicates a milk column M3 with a low macronutrient value, the curve b indicates a milk column M3 with a moderate macronutrient value, and the curve c indicates a milk column M3 with a high macronutrient value. This can be seen from the worsening flow behaviour of the milk.

It is also possible to determine the number of milk columns M3 that have passed through during a time interval. The frequency of the throughflow of the milk columns can also be determined. Moreover, other methods are possible for determining the viscosity of the milk columns and therefore the macronutrient value.

FIGS. 8 to 14 show further examples of sensor units 3 according to the invention, which can be used like the above-described sensor unit 3 in the described breastshield unit or in other regions of the breast pump. The milk channel section 63 has in each case the same channel regions and siphon bends as in the first example, although not all of these elements are visible in the figures.

In the illustrative embodiment according to FIG. 8, the main body 30 of the sensor unit 3 has a substantially circular cylindrical shape. The cover 34 of the milk channel section 36 is designed as a half shell. The lower attachment part 32 has an upwardly directed shoulder 321, on which the cover 34 lies.

In the embodiment according to FIG. 9, the inlet 35 has a smaller diameter than in the previous examples. The part of the milk channel section 36 designed as a groove is formed in a plane surface and not, as in the previous examples, on a curved jacket region. The cover 34 accordingly has a segment-shaped cross section and is likewise plane in the direction towards the main body 30.

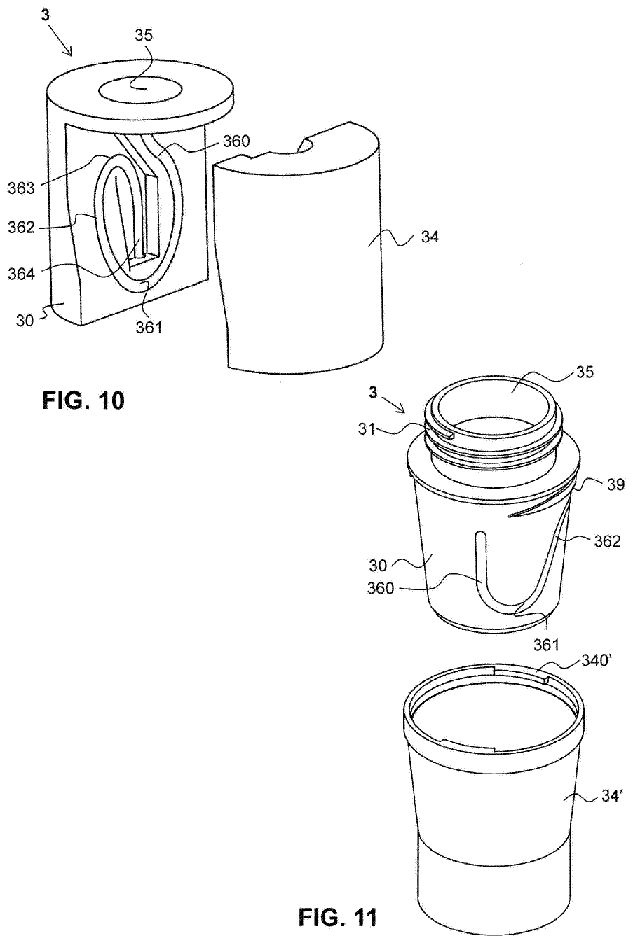

In the illustrative embodiment according to FIG. 10, the main body 30 and the cover 34 are provided with steps. The siphon is configured such that the upper siphon bend 363 extends at least partially perpendicularly with respect to the lower siphon bend 361.

FIG. 11 shows the cover 34′ designed as a sleeve which is engaged over the main body 30, wherein the two components latch onto each other by means of closure elements 340′, 39.

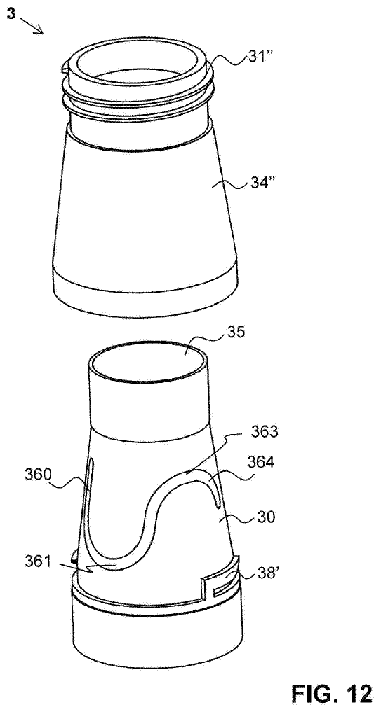

FIG. 12 shows the first embodiment described with reference to FIGS. 1 to 5. This breastshield unit has a sleeve as cover 34″ which, in contrast to the previously described example, is engaged over the main body 30 from above.

In these examples, the groove of the milk channel section 36 is always arranged on the outer surface of the main body. It can also be arranged additionally or alternatively in the cover.

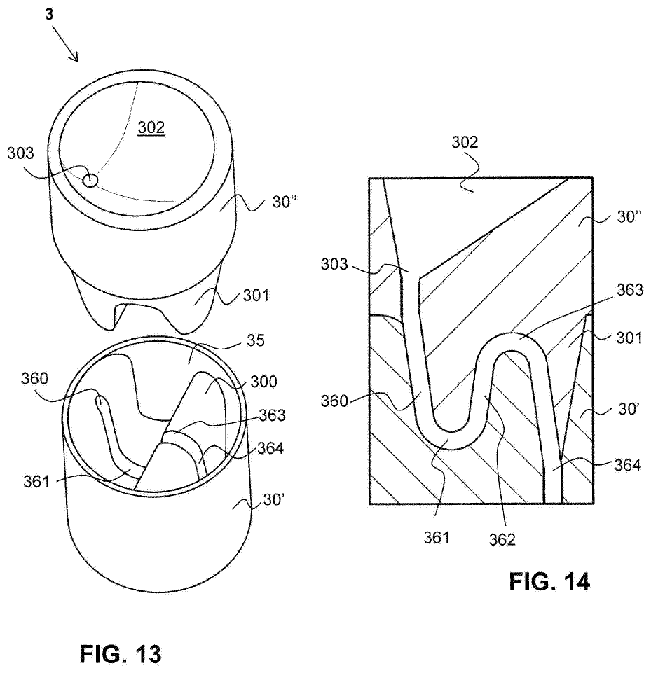

In the illustrative embodiment according to FIGS. 13 and 14, the milk channel section 36 is formed entirely in the interior of the sensor unit 3. A lower main body 30′ has a lower inner undulating structure 300, along the surface of which the upwardly open groove of the milk channel section 36 extends. An upper main body 30″ serves as a cover and has an upper undulating structure 301, which forms the counterpart to the lower undulating structure 300. Moreover, the upper main body 30″ preferably has an inlet with an inclined surface 302 and with an off-centre inlet opening 303, which forms the access to the siphon-like milk channel section 36. FIG. 14 shows the upper main body 30″ and lower main body 30′ in the assembled state, as a result of which the milk channel section again has a closed configuration, except for its inlet and its outlet.

All of these illustrative embodiments permit optimal placement of the sensors and of the electronics unit and, by virtue of the groove-like siphon and the removable cover, they permit simple and thorough cleaning.

The breastshield unit according to the invention permits optimal measurement of properties of the expressed breastmilk.

LIST OF REFERENCE SIGNS

| | | | |
|---|---|---|---|
| 1 | breastshield | 361 | lower siphon bend |
| 10 | shield | 362 | second channel region |
| 11 | connector | 363 | upper siphon bend |
| 12 | expression region | 364 | third channel region |
| | | 365 | fourth channel region |
| 2 | breastshield adapter | 366 | sealing lip |
| 20 | main body | 37 | outlet |
| 21 | attachment | 38 | cavity |
| 22 | valve | 39 | second closure element |
| | | 39′ | closure element |
| 3 | sensor unit | | |
| 30 | main body | 4 | milk collection container |
| 30′ | lower main body | 40 | main body |
| 30″ | upper main body | 41 | neck |
| 300 | lower undulating structure | | |
| 301 | upper undulating | 5 | electronics unit |
| | structure | 50 | battery |
| 302 | inclined surface | 51 | reed switch |
| 303 | inlet opening | 52 | microcontroller |
| 304 | outlet opening | 53 | printed circuit board |
| 31 | upper attachment part | 54 | acceleration sensor |
| 31′ | upper attachment part | | |
| 32 | lower attachment part | 6 | first light barrier |
| 320 | inner thread | | |
| 321 | shoulder | 7 | second light barrier |
| 33 | holding element | | |
| 34 | cover | M1 | expressed milk |
| 34′ | lower sleeve | M2 | milk lake |
| 34″ | upper sleeve | M3 | milk column |
| 340′ | first closure element | | |
| 35 | inlet | T1 | first separation line |
| 36 | milk channel section | T2 | second separation line |
| 360 | first channel region | | |

1. A breastshield unit of a breast pump for expressing human breastmilk, wherein the breastshield unit comprises a breastshield for placing onto a human breast, a milk collection container for receiving expressed breastmilk, a breastshield adapter for connecting the breastshield to the milk collection container, and at least one sensor for detecting the breastmilk, wherein a milk channel extends from the breastshield through the breastshield adapter into the milk collection container, wherein the milk channel defines a direction of flow of the breastmilk, wherein a siphon is arranged in the milk channel, wherein the at least one sensor for detecting the breastmilk is arranged downstream from the siphon in the direction of flow of the breastmilk.

2. The breastshield unit according to claim 1, wherein a valve is present which closes and opens the milk channel during the expression process, and wherein the siphon in the milk channel is arranged downstream from the valve in the direction of flow of the breastmilk.

3. The breastshield unit according to claim 2, wherein the milk channel comprises a widened region between the valve and the siphon.

4. The breastshield unit according to claim 1, wherein it comprises a signal evaluation module for evaluating signals of the at least one sensor, wherein the signal evaluation module supplies a profile of the signals as a function of time, as a result of which at least one of the following variables can be determined: throughflow quantity, throughflow rate, throughflow velocity, macronutrients, and fat content.

5. The breastshield unit according to claim 1, wherein a sensor unit is present, wherein the sensor unit comprises the siphon of the milk channel and the at least one sensor.

6. The breastshield unit according to claim 5, wherein the sensor unit comprises a main body with a surface and with a cover tightly closing this surface, wherein at least the siphon of the milk channel is formed by a groove which is configured in at least one of the surface and the cover and which, by the cooperation of the surface with the cover, is tightly closed except for an inlet and an outlet.

7. The breastshield unit according to claim 6, wherein the surface is a radially outwardly oriented surface of the main body.

8. The breastshield unit according to claim 7, wherein the cover is one of a lid or a sleeve.

9. The breastshield unit according to claim 6, wherein the sensor unit comprises an interior which is open at the top and in which the surface is formed in an undulating shape, and wherein the cover is configured as an undulating counterpart.

10. The breastshield unit according to claim 6, wherein the main body comprises a cavity for receiving an electronics unit.

11. The breastshield unit according to claim 10, wherein the electronics unit comprises the signal evaluation module.

12. The breastshield unit according to claim 5, wherein the sensor unit has the shape of a circular cylinder or the shape of a truncated cone.

13. The breastshield unit according to claim 1, wherein the at least one sensor is at least one optical sensor.

14. The breastshield unit according to claim 1, wherein more than one sensor is present, and wherein different sensors are present.

15. A sensor unit of a breast pump for expressing human breastmilk, wherein the breast pump comprises a breastshield for placing onto a human breast, a milk collection container for receiving expressed breastmilk, a milk channel extending from the breastshield to the milk collection container, and at least one sensor for detecting the breastmilk, wherein the milk channel defines a direction of flow of the breastmilk, the sensor unit comprising a part of the milk channel, wherein this part of the milk channel forms a siphon, and the at least one sensor for detecting the breastmilk being arranged in the sensor unit downstream from the siphon in the direction of flow of the breastmilk.

16. A method for determining at least one property of human breastmilk during expression of the human breastmilk, wherein the breastmilk is expressed by application of a changing negative pressure, wherein the method comprises:
collecting the breastmilk in a damming region of a siphon region,
emptying the damming region batch by batch via a siphon loop of the siphon region to form a breastmilk column,
forwarding the breastmilk column to at least one sensor,
detecting the breastmilk column, which has been forwarded batch by batch, by means of the at least one sensor, and
evaluating signals of the sensor as a function of time.

17. The breastshield unit according to claim 5, wherein the sensor unit is arranged between the breastshield adapter and the milk collection container.
